(19) United States
(12) Patent Application Publication
(10) Pub. No.: US 2013/0178853 A1
(43) Pub. Date: Jul. 11, 2013

(54) SURGICAL TOOL MANAGEMENT

(71) Applicants: Carolyn A. Hyink, Austin, TX (US); Peter F. Haggar, Raleigh, NC (US); Ashwin B. Manekar, Morrisville, NC (US); Artem A. Papkov, Apex, NC (US)

(72) Inventors: Carolyn A. Hyink, Austin, TX (US); Peter F. Haggar, Raleigh, NC (US); Ashwin B. Manekar, Morrisville, NC (US); Artem A. Papkov, Apex, NC (US)

(73) Assignee: INTERNATIONAL BUSINESS MACHINES CORPORATION, Armonk, NY (US)

(21) Appl. No.: 13/344,254

(22) Filed: Jan. 5, 2012

(51) Int. Cl. A61B 17/00

(52) U.S. Cl. 606/79; 606/1

(57) ABSTRACT

The different illustrative embodiments provide a method, computer program product, and apparatus for managing a number of surgical tools. A determination is made whether a number of surgical tools is to be used in performing a procedure based on information. Responsive to a determination that the number of surgical tools is to be used in performing the procedure, a determination is made whether a signal received by the number of surgical tools indicates that the number of surgical tools is present within a predetermined distance of a location on the patient where the procedure is to be performed. Responsive to a determination that the signal received by the number of surgical tools indicates that the number of surgical tools is within the predetermined distance of the location on the patient where the procedure is to be performed, the number of surgical tools is enabled for use in performing the procedure.

### BACKGROUND

1. Field

The disclosure relates generally to an improved data processing system and more specifically to managing a surgical environment in an improved data processing system. More specifically, the disclosure relates to a method, computer program product, and apparatus for managing a number of surgical tools.

2. Description of the Related Art

In medicine, surgery is a procedure in which a number of medical personnel perform a modification to a portion of the body of a patient. For example, the surgery may be performed to rework an inconsistency in the body of the patient, such as an operation to remove plaque from arteries of the patient. In another example, surgery may be performed to add a medical device to the body of the patient and/or replace a medical device in the body of the patient, such as a pacemaker. In yet another example, surgery may be performed to make a desired cosmetic change to the body of the patient, such as reworking one or more portions of the face of a patient.

Before the surgery is performed, medical personnel perform a number of processes to prepare the patient for surgery. The processes include selecting a site on the body of the patient at which to perform the surgery. The site of the surgery may be selected by the number of medical personnel based on the procedure to be performed. For example, a doctor may select a site on the chest of a patient to perform surgery to add a pacemaker to the body of the patient. The site selected for surgery to rework the spine may be the lower back of the patient, in another example.

In some illustrative examples, a doctor or other medical personnel select the site for the surgery prior to the surgery. For example, the medical personnel may select the site for the surgery hours or days before the surgery is performed. In such illustrative examples, the medical personnel may make a record of the site at which the surgery is to be performed. For example, the medical personnel may write a note in the file of the patient where the surgery is to be performed.

At another time prior to the surgery being performed, a number of medical personnel may prepare the body of the patient for surgery. For example, the number of medical personnel may remove hair from the site and/or apply treatments to the site to reduce undesirable entry of microbes into the body of the patient. The number of medical personnel may also read the file of the patient and mark the site of the surgery to be performed using the information in the file. For example, a nurse may draw on the site using a marker.

Such a marking may be made while a doctor performing the surgery is making other preparations, such as dressing in surgical clothing, scrubbing the hands of the doctor, or another suitable process. The personnel to perform the surgery then locate the marking representing the site where the surgery is to be performed and begin the procedure on the patient. For example, the number of medical personnel may then locate the marking on the right elbow of the patient and use a number of surgical tools to open the arm and perform the desired surgical procedure.

### SUMMARY

The different illustrative embodiments provide a method, computer program product, and apparatus for managing a number of surgical tools. Information transmitted by a tag associated with a patient is received about a procedure to be performed on the patient. A determination is made as to whether a number of surgical tools is to be used in performing the procedure based on the information. Responsive to a determination that the number of surgical tools is to be used in performing the procedure, a determination is made as to whether a signal received by the number of surgical tools indicates that the number of surgical tools is present within a predetermined distance of a location on the patient where the procedure is to be performed. Responsive to a determination that the signal received by the number of surgical tools indicates that the number of surgical tools is within the predetermined distance of the location on the patient where the procedure is to be performed, the number of surgical tools is enabled for use in performing the procedure.

### BRIEF DESCRIPTION OF THE SEVERAL VIEWS OF THE DRAWINGS

FIG. 1 depicts an illustration of a surgical environment in accordance with an illustrative embodiment;

FIG. 2 depicts a diagram of a data processing system in accordance with an illustrative embodiment;

FIG. 3 depicts an illustration of a surgical tool management environment in accordance with an illustrative embodiment;

FIG. 4 depicts an illustration of a cutting tool in a disabled state in accordance with an illustrative embodiment;

FIG. 5 depicts an illustration of a cutting tool in an enabled state in accordance with an illustrative embodiment;

FIG. 6 depicts an illustration of a screenshot of a confirmation interface in accordance with an illustrative embodiment;

FIG. 7 depicts an illustration of a screenshot of a second confirmation interface in accordance with an illustrative embodiment;

FIG. 8 depicts an illustration of a flowchart of a process for managing a number of surgical tools in accordance with an illustrative embodiment;

FIG. 9 depicts an illustration of a flowchart of a process for associating a tag having an identifier with a surgical plan in accordance with an illustrative embodiment;

FIG. 10 depicts an illustration of a flowchart of a process for managing a surgical procedure in accordance with an illustrative embodiment; and

FIG. 11 depicts an illustration of a process for managing a surgical tool in accordance with an illustrative embodiment.

### DETAILED DESCRIPTION

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

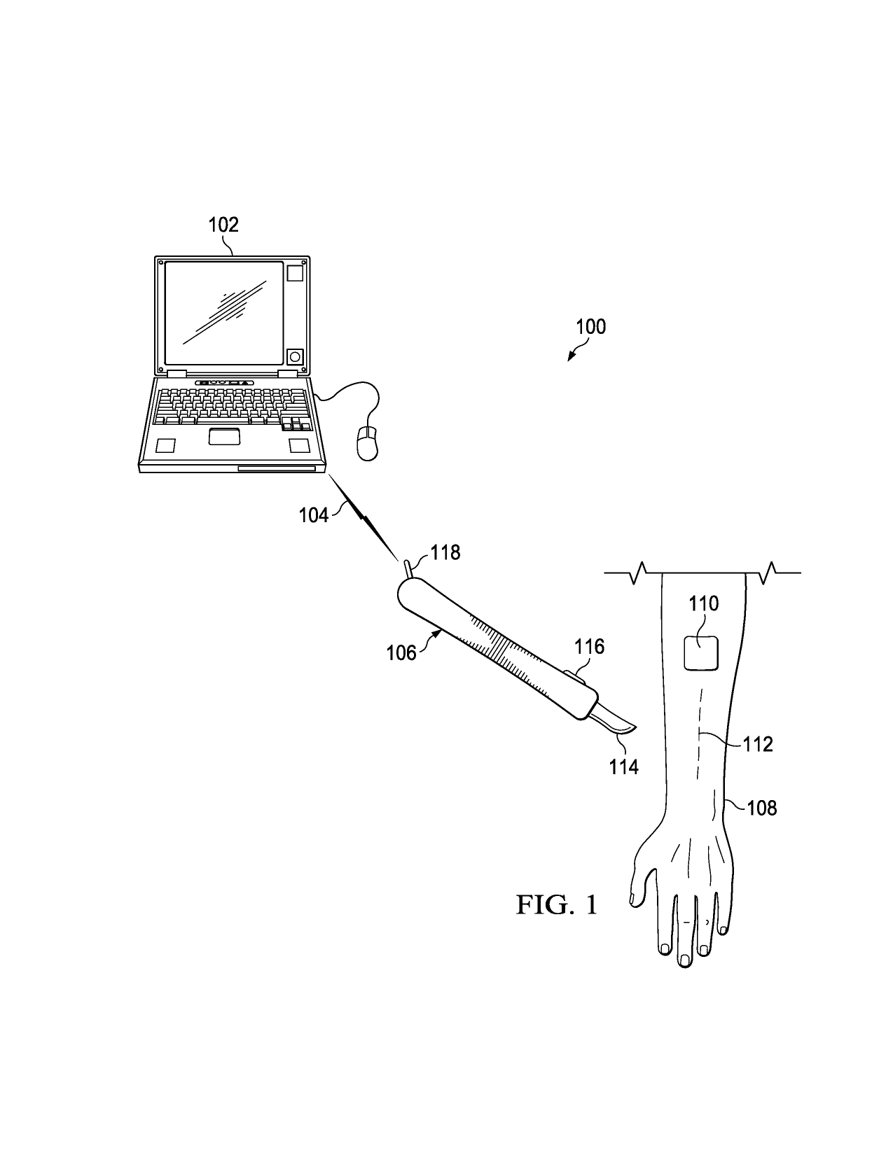

With reference now to FIG. 1, an illustration of a surgical environment is depicted in accordance with an illustrative embodiment. Surgical environment 100 is an example of an environment in which illustrative embodiments may be implemented.

Surgical environment 100 includes computer system 102, link 104, surgical tool 106, patient 108, and tag 110. Computer system 102 communicates with surgical tool 106 using link 104. Link 104 is a data communication channel. For example, link 104 may be a wired or wireless network. Surgical tool 106 is an item used to perform a surgical procedure on patient 108. For example, surgical tool 106 is a scalpel in this illustrative embodiment.

In this illustrative embodiment, medical personnel desire to perform a surgical procedure on patient 108. One step in the procedure may be to make an incision along line 112. Prior to the surgical procedure, medical personnel removably attach tag 110 to patient 108 at or near the site at which the surgical procedure will be performed. Tag 110 is a data storage system that includes a communications unit. Tag 110 transmits at least a portion of the data stored in the data storage system using the communications unit. For example, tag 110 may be a radio frequency identification tag. In such an illustrative embodiment, tag 110 may transmit information stored in a memory associated with tag 110 using radio waves.

When the surgical procedure is being performed, surgical tool 106 is disabled. In other words, surgical tool 106 may not be used to perform the function of surgical tool 106 on patient 108. In this illustrative example, the scalpel is disabled by blade 114 being retracted into the housing of surgical tool 106. In one illustrative example, surgical tool 106 engages a motor to retract blade 114. When surgical tool 106 is held within a transmission range of tag 110, surgical tool 106 receives information transmitted by tag 110 using receiver 116. In this illustrative example, receiver 116 is a radio frequency identification tag receiver. The information may include an identifier for the patient and/or the procedure to be performed, an identification of the surgical tools to be used to perform the procedure, or other suitable information.

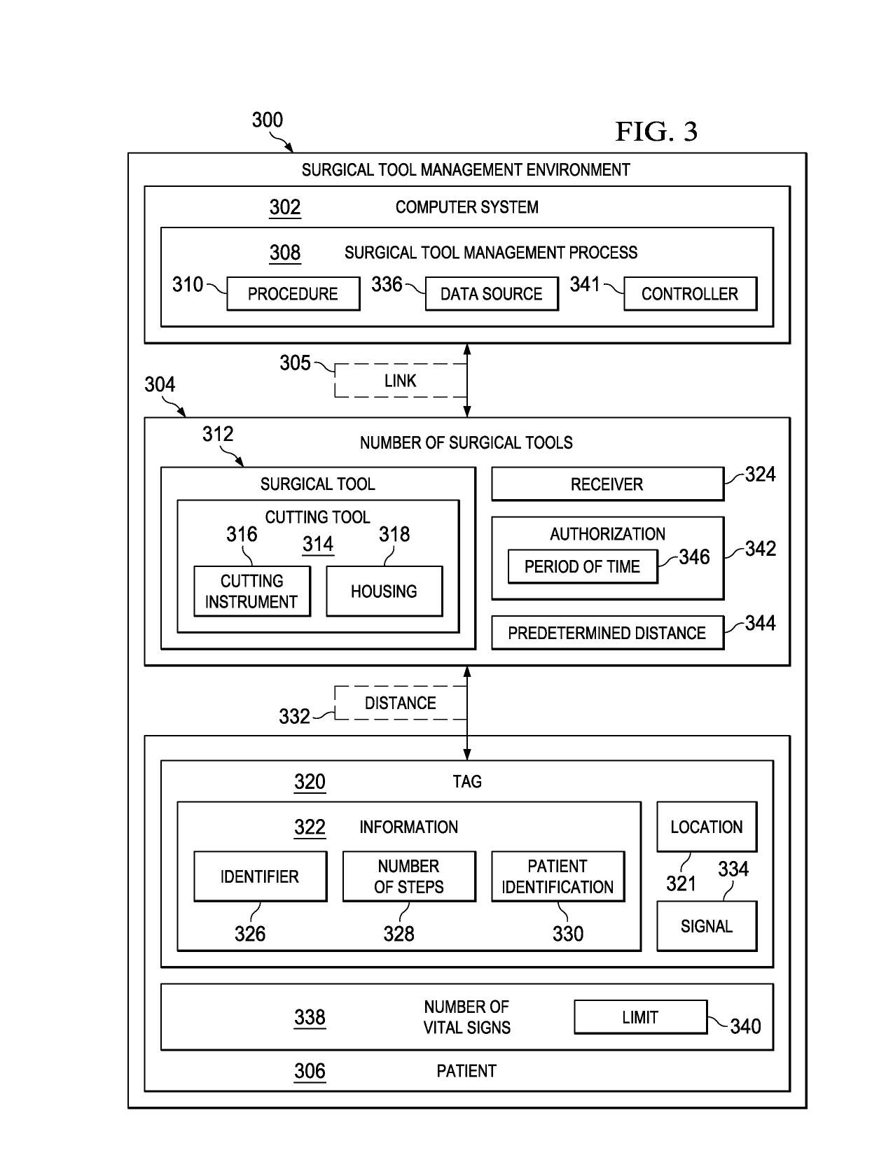

When surgical tool 106 receives the information from tag 110, surgical tool 106 transmits the information to computer system 102 using link 104. In this illustrative embodiment, surgical tool 106 transmits the information to computer system 102 wirelessly using antenna 118. A process running on computer system 302, such as surgical tool management process 308 in FIG. 3, receives the information. In some illustrative embodiments, surgical tool 106 also identifies a distance between surgical tool 106 and tag 110. For example, surgical tool 106 may identify the distance by identifying the strength of the signal being transmitted by tag 110.

The process running on computer system 102 then determines whether surgical tool 106 is to be used in performing the surgical procedure. The process may determine whether surgical tool 106 is to be used by identifying a list of tools for the procedure based on the information received from surgical tool 106 and/or information stored in a data source accessible to computer system 102. In some illustrative examples, the process running on computer system 102 also determines whether the distance received from surgical tool 106 is less than a predetermined distance. The predetermined distance is a distance from the tag within which enabling of surgical tool 106 is desired.

When the process determines that surgical tool 106 is to be used to perform the surgical procedure and surgical tool 106 is within the predetermined distance, the process causes computer system 102 to transmit an authorization to surgical tool 106. Surgical tool 106 receives the authorization and is enabled for a period of time. In this illustrative embodiment, surgical tool 106 is enabled by extending blade 114 from within the housing of surgical tool 106 such that blade 114 may contact patient 110. Surgical tool 106 continues to transmit the information and/or the distance to computer system 102.

The process running on computer system 102 continues to cause computer system 102 to transmit authorization to surgical tool 106 when surgical tool 106 is to be used to perform the procedure and surgical tool 106 is within the predetermined distance of tag 110. Of course, such transmissions may occur continuously, on a predetermined interval, or another suitable schedule in the different illustrative embodiments. In the event that surgical tool 106 does not receive an authorization within a predetermined amount of time, surgical tool 106 is disabled. In this illustrative embodiment, surgical tool 106 retracts blade 114 into the housing of surgical tool 106 such that blade 114 may not contact patient 108.

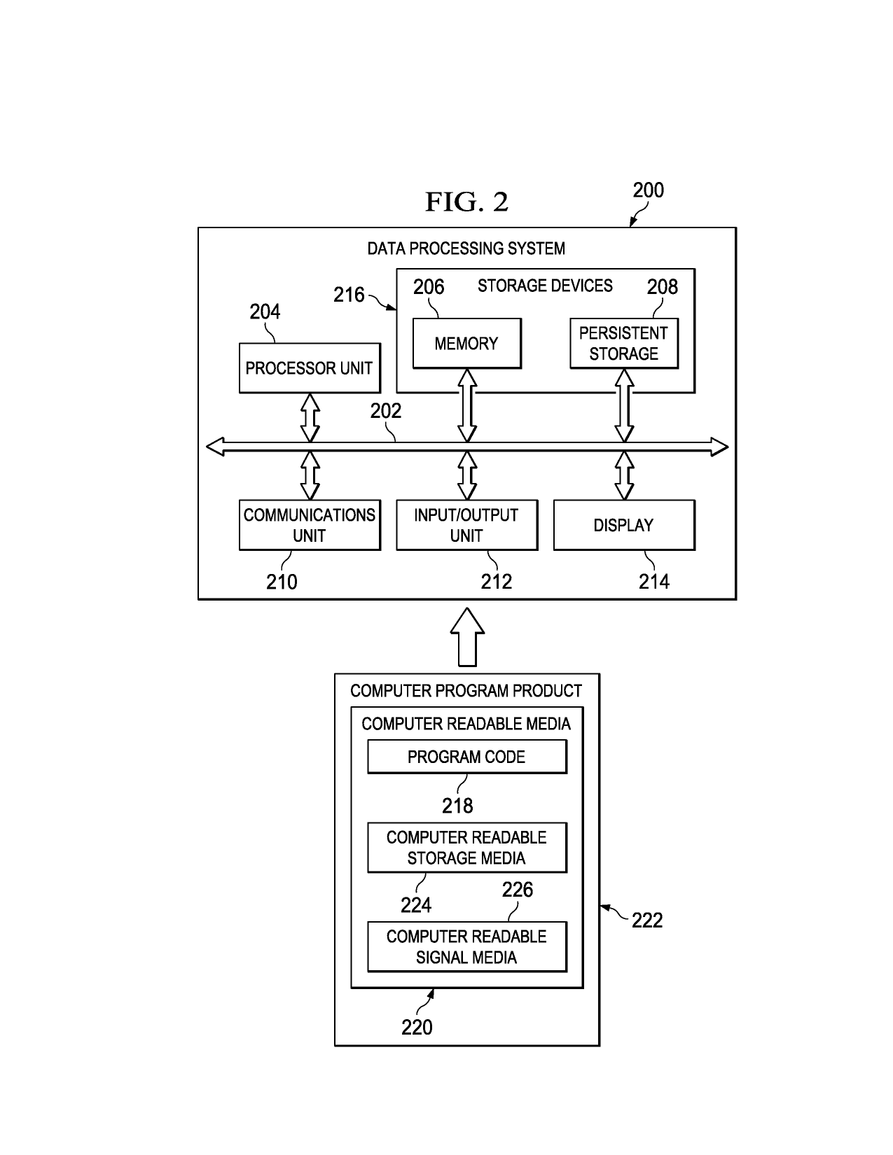

Turning now to FIG. 2, a diagram of a data processing system is depicted in accordance with an illustrative embodiment. In this illustrative example, data processing system 200 includes communications fabric 202, which provides communications between processor unit 204, memory 206, persistent storage 208, communications unit 210, input/output (I/O) unit 212, and display 214. Data processing system 200 is an example of a data processing system that can be used to implement server computers and client computers in network data processing system 100 in FIG. 1. More specifically, in one illustrative example, data processing system 200 may run a surgical tool management process, such as surgical tool management process 308 in FIG. 3.

Processor unit 204 serves to process instructions for software that may be loaded into memory 206. Processor unit 204 may be a number of processors, a multi-processor core, or some other type of processor, depending on the particular implementation. A “number”, as used herein, with reference to an item, means “one or more items”. Further, processor unit 204 may be implemented using a number of heterogeneous processor systems in which a main processor is present with secondary processors on a single chip. As another illustrative example, processor unit 204 may be a symmetric multi-processor system containing multiple processors of the same type.

Memory 206 and persistent storage 208 are examples of storage devices 216. A storage device is any piece of hardware that is capable of storing information, such as, for example without limitation, data, program code in functional form, and/or other suitable information either on a temporary basis and/or a permanent basis. Memory 206, in these examples, may be, for example, a random access memory or any other suitable volatile or non-volatile storage device. Persistent storage 208 may take various forms depending on the particular implementation. For example, persistent storage 208 may contain one or more components or devices. For example, persistent storage 208 may be a hard drive, a flash memory, a rewritable optical disk, a rewritable magnetic tape, or some combination of the above. The media used by persistent storage 208 also may be removable. For example, a removable hard drive may be used for persistent storage 208.

Communications unit 210, in these examples, provides for communications with other data processing systems or devices. In these examples, communications unit 210 is a network interface card. Communications unit 210 may provide communications through the use of either or both physical and wireless communications links.

Input/output unit 212 allows for input and output of data with other devices that may be connected to data processing system 200. For example, input/output unit 212 may provide a connection for user input through a keyboard, a mouse, and/or some other suitable input device. Further, input/output unit 212 may send output to a printer. Display 214 provides a mechanism to display information to a user.

Instructions for the operating system, applications and/or programs may be located in storage devices 216, which are in communication with processor unit 204 through communications fabric 202. In these illustrative examples, the instructions are in a functional form on persistent storage 208. These instructions may be loaded into memory 206 for execution by processor unit 204. The processes of the different embodiments may be performed by processor unit 204 using computer implemented instructions, which may be located in a memory, such as memory 206.

These instructions are referred to as program code, computer usable program code, or computer readable program code that may be read and processed by a processor in processor unit 204. The program code in the different embodiments may be embodied on different physical or computer readable storage media, such as memory 206 or persistent storage 208.

Program code 218 is located in a functional form on computer readable media 220 that is selectively removable and may be loaded onto or transferred to data processing system 200 for execution by processor unit 204. Program code 218 and computer readable media 220 form computer program product 222 in these examples. In one example, computer readable media 220 may be computer readable storage media 224 or computer readable signal media 226. Computer readable storage media 224 may include, for example, an optical or magnetic disc that is inserted or placed into a drive or other device that is part of persistent storage 208 for transfer onto a storage device, such as a hard drive that is part of persistent storage 208. Computer readable storage media 224 also may take the form of a persistent storage, such as a hard drive, a thumb drive, or a flash memory that is connected to data processing system 200. In some instances, computer readable storage media 224 may not be removable from data processing system 200. In these illustrative examples, computer readable storage media 224 is a non-transitory computer readable storage media.

Alternatively, program code 218 may be transferred to data processing system 200 using computer readable signal media 226. Computer readable signal media 226 may be, for example, a propagated data signal containing program code 218. For example, computer readable signal media 226 may be an electro-magnetic signal, an optical signal, and/or any other suitable type of signal. These signals may be transmitted over communications links, such as wireless communications links, optical fiber cable, coaxial cable, a wire, and/or any other suitable type of communications link. In other words, the communications link and/or the connection may be physical or wireless in the illustrative examples.

In some illustrative embodiments, program code 218 may be downloaded over a network to persistent storage 208 from another device or data processing system through computer readable signal media 226 for use within data processing system 200. For instance, program code stored in a computer readable storage medium in a server data processing system may be downloaded over a network from the server to data processing system 200. The data processing system providing program code 218 may be a server computer, a client computer, or some other device capable of storing and transmitting program code 218.

The different components illustrated for data processing system 200 are not meant to provide architectural limitations to the manner in which different embodiments may be implemented. The different illustrative embodiments may be implemented in a data processing system including components in addition to or in place of those illustrated for data processing system 200. Other components shown in FIG. 2 can be varied from the illustrative examples shown. The different embodiments may be implemented using any hardware device or system capable of executing program code. As one example, the data processing system may include organic components integrated with inorganic components, and/or may be comprised entirely of organic components, excluding a human being. For example, a storage device may be comprised of an organic semiconductor.

As another example, a storage device in data processing system 200 is any hardware apparatus that may store data. Memory 206, persistent storage 208 and computer readable media 220 are examples of storage devices in a tangible form.

In another example, a bus system may be used to implement communications fabric 202 and may be comprised of one or more buses, such as a system bus or an input/output bus. Of course, the bus system may be implemented using any suitable type of architecture that provides for a transfer of data between different components or devices attached to the bus system. Additionally, a communications unit may include one or more devices used to transmit and receive data, such as a modem or a network adapter. Further, a memory may be, for example, memory 206 or a cache, such as found in an interface and memory controller hub that may be present in communications fabric 202.

The different illustrative embodiments recognize and take into account a number of different considerations. For example, the different illustrative embodiments recognize and take into account that the surgical environment is complex. More specifically, the surgical environment may include multiple medical personnel that are only involved in the surgical procedure for a portion of the preparation and performance of the procedure.

For example, a nurse that prepares the site on the body of the patient for surgery, including marking and/or drawing on the site, may not have been present for the selection of the site by the doctor that is to perform the surgery. The nurse may also be absent for the performance of the surgical procedure. Further, the doctor may make an illegible or inconsistent note in the file of the patient with reference to the site of the procedure to be performed. In some illustrative examples, a period of hours or days elapses between the selection of the site by the doctor and the performance of the procedure.

The different illustrative embodiments recognize and take into account that the complexity of the surgical environment may cause inconsistencies to develop in the performance of the surgical procedure. For example, the different illustrative embodiments recognize and take into account that a surgical procedure may be performed on an undesired site on the body of the patient. One or more of the medical personnel may record, mark, or perform the procedure on a site on the body of the patient other than the desired site.

Thus, the different illustrative embodiments allow a number of medical personnel to select the site for a surgical procedure to be performed together with a patient prior to the preparation for surgery and receive confirmation of the desired site for the procedure. For example, a doctor may explain the surgery, demonstrate the site for the surgery to be performed to the patient, and request the confirmation of the patient for the site of the procedure.

A tag may then be removably attached at or near the site selected by the doctor and patient. The tag may include digital information, such as an identifier for the patient, information about the surgical procedure to be performed, information about the confirmation of the patient for the site, and other suitable information. The tag has a communication system in which the information on the tag may be read wirelessly. For example, the tag may be a radio frequency identification tag.

The different illustrative embodiments also recognize and take into account that the number of surgical tools used by the number of medical personnel to perform the surgery may be enabled when a signal is received from a controller. The number of surgical tools may have a tag reading system that reads the data from the tag and sends the data from the tag to the controller. The data may also include an identification of the surgical tool sending the data. The number of surgical tools may read the data from the tag and send the data to the controller once, continuously, or at a predetermined frequency.

The controller receives the data and determines that a number of operational conditions are met. For example, the controller may send a wireless signal to the number of surgical tools that causes the surgical tools to be enabled when the surgical tool from which the tag data was received is within a predetermined range of the tag and/or the vital signs of the patient are within predetermined bounds. For example, a scalpel may extend the blade of the scalpel from within a housing when the scalpel receives the signal from the controller.

The controller may cease sending the signal that enables one or more of the number of surgical tools when one or more operational conditions are not met. For example, when a surgical tool is moved outside the predetermined range of the tag and no longer sends the data from the tag to the controller, the controller may cease sending the signal that enables the surgical tool. When the surgical tool has not received the signal for a predetermined amount of time, the surgical tool is disabled. For example, a scalpel may retract the blade of the scalpel into a housing.

Thus, the different illustrative embodiments provide a method, computer program product, and apparatus for managing a number of surgical tools. Information transmitted by a tag associated with a patient is received about a procedure to be performed on the patient. A determination is made as to whether a number of surgical tools is to be used in performing the procedure based on the information. Responsive to a determination that the number of surgical tools is to be used in performing the procedure, a determination is made as to whether a signal received by the number of surgical tools indicates that the number of surgical tools is present within a predetermined distance of a location on the patient where the procedure is to be performed. Responsive to a determination that the signal received by the number of surgical tools indicates that the number of surgical tools is within the predetermined distance of the location on the patient where the procedure is to be performed, the number of surgical tools is enabled for use in performing the procedure.

Turning now to FIG. 3, an illustration of a surgical tool management environment is depicted in accordance with an illustrative embodiment. Surgical tool management environment 300 is an example of an environment in which illustrative embodiments may be implemented. Surgical tool management environment 300 includes computer system 302, number of surgical tools 304, and patient 306 in this illustrative embodiment.

Computer system 302 is an example implementation of computer system 200 in FIG. 2. In these illustrative examples, computer system 302 is one computer system. However, in other illustrative examples, computer system 302 may be a number of computer systems. As used herein, “a number of items” mean one or more of the items. For example, a number of computer systems mean one or more computer systems.

Computer system 302 runs surgical tool management process 308. For example, surgical tool management process 308 may be program instructions that are run by a processor unit associated with computer system 302. Surgical tool management process 308 receives data from number of surgical tools 304 and/or transmits data to number of surgical tools 304. The data may be received over a wired or wireless link, such as link 305. For example, the data may be received using a wireless networking transmission, such as IEEE 802.11n, radio transmission, such as frequency modulation transmission, or another suitable wireless data transmission method.

Number of surgical tools 304 is a set of items used to perform procedure 310 on patient 306. For example, number of surgical tools 304 may include a scalpel, a saw, and/or another suitable tool. Procedure 310 is a medical procedure that is performed on the physical body of patient 306. For example, procedure 310 may be adding a pacemaker to the body of patient 306.

Procedure 310 is a surgical procedure in these illustrative examples. A surgical procedure is a procedure performed in which a number of medical professionals perform a modification on the body of patient 306. For example, procedure 310 may include removing plaque from the vascular system of patient 306, adding a pacemaker to the body of patient 306, reworking an inconsistency in the face of patient 306, or another suitable procedure.

Surgical tool 312 in number of surgical tools 304 is cutting tool 314 in this illustrative embodiment. Cutting tool 314 is a device having cutting instrument 316 and housing 318. More specifically, cutting tool 314 may be a scalpel, a saw, or another suitable tool that cuts the body of patient 306 when used on the body of patient 306. In this illustrative embodiment, cutting tool 314 is a scalpel. Thus, cutting instrument 316 may be a blade associated with housing 318.

Cutting instrument 316 of cutting tool 314 is configured to extend from housing 318 when cutting instrument 316 is enabled. Cutting instrument 316 of cutting tool 314 is further configured to retract into housing 318 when cutting instrument 316 is disabled. In this illustrative embodiment, cutting tool 314 moves cutting instrument 316 to a location within housing 318 such that cutting instrument 316 may not contact patient 306. When cutting instrument 316 is extended, cutting instrument 316 may contact patient 306 and cut the body of patient 306.

Computer system 302 also receives data from tag 320. Tag 320 is removably attached to the body of patient 306 at location 321. Location 321 is a point or area near which procedure 310 is to be performed. In these illustrative embodiments, computer system 302 receives information 322 from tag 320. Information 322 is stored in memory associated with tag 320 and is transmitted using a wired or wireless transmission. For example, tag 320 may be a radio frequency identification tag. In such an illustrative example, information 322 may be transmitted to receiver 324 associated with number of surgical tools 304. Receiver 324 is a radio frequency identification tag receiver in this illustrative example. Number of surgical tools 304 may then transmit information 322 received from tag 320 to computer system 302 using link 305.

Information 322 includes data used to prepare for procedure 310 and/or to perform procedure 310. For example, information 322 may include identifier 326. Identifier 326 is a number or other identifying value that represents procedure 310. For example, identifier 326 may be an identification number of procedure 310. Information 322 may also include number of steps 328. Number of steps 328 are tasks to be done while performing procedure 310. For example, number of steps 328 may include a list of the areas to be cut, the items to be added or removed from the patient, or other suitable steps. Information 322 may also include patient identification 330. Patient identification 330 is information about the identity of patient 306. For example, patient identification 330 may include the name of the patient and/or a patient identification number.

Number of surgical tools 304 may also identify distance 332 between number of surgical tools 304 and tag 320. Number of surgical tools 304 may use strength of signal 334 transmitted by tag 320 and received by receiver 324 of number of surgical tools to identify distance 332. In one illustrative example, signal 334 includes information 322. In another illustrative example, information 332 is transmitted to receiver 324 in another transmission.

Thus, in an illustrative embodiment in which tag 320 is a radio frequency identification tag, tag 320 may transmit signal 334 that includes information 322 and is received by receiver 324 of number of surgical tools 304. Number of surgical tools 304 may then transmit information 322 and information about distance 332 to computer system 302. The information about distance 332 may be a linear distance, a strength of signal 334 received by receiver 324, or another suitable representation. Transmitting and receiving signal 334 and transmitting information 322 to computer system 302 may be performed once, continuously, on a schedule, or another suitable time period.

Computer system 302 receives information 322 and distance 332 from number of surgical tools 304. Surgical tool management process 308 then determines whether number of surgical tools 304 is to be used in performing procedure 310 based on information 322. For example, surgical tool management process 308 may use identifier 326 for procedure 310 to retrieve an identification of tools used in procedure 310 from data source 336. Surgical tool management process 308 may then determine whether each of number of surgical tools 304 is to be used in procedure 310.

In another illustrative embodiment, surgical tool management process 308 may receive number of steps 328 in information 322. Surgical tool management process 308 may then determine whether number of surgical tools 304 are to be used in performing number of steps 328 using information about number of surgical tools 304 and/or number of steps 328 in data source 336. In other words, surgical tool management process 308 running on computer system 304 is controller 341 for number of surgical tools 304.

In yet another illustrative embodiment, surgical tool management process 308 determines whether patient identification 330 matches the identification of a patient in data source 336 that is to have procedure 310 performed at the present time. In another illustrative embodiment, surgical tool management process 308 determines that number of surgical tools 304 is not to be used when number of vital signs 338 for patient 306 is not within limit 340. Number of vital signs 338 is statistics related to the bodily functions of patient 306. For example, number of vital signs may include a pulse and an oxygen saturation level. Limit 340 is a threshold for each of number of vital signs 338.

In some illustrative embodiments, surgical tool management process 308 may only determine that number of surgical tools 304 is to be used to perform procedure 310 when number of surgical tools 304 and distance 332 indicates that number of surgical tools 304 is located within predetermined distance 344 of tag 320. Predetermined distance 344 is an amount of distance between tag 320 and number of surgical tools 304 that indicates when number of surgical tools 304 is likely to be used at location 321 on the body of patient 306.

In the event that surgical tool management process 308 determines that number of surgical tools 304 are to be used in performing procedure 310, surgical tool management process 308 causes number of surgical tools 304 to be enabled. In these illustrative examples, surgical tool management process 308 transmits authorization 342 to number of surgical tools 304 using link 305. Number of surgical tools 304 receives authorization 342 from computer system 302 running surgical tool management process 308 and enables number of surgical tools 304. For example, cutting tool 314 is enabled by extending cutting instrument 316 outside of housing 318 such that cutting instrument 316 may contact patient 306.

Once number of surgical tools 304 is enabled, number of surgical tools 304 continues to receive information 322 from tag 320. In these illustrative embodiments, receiver 324 receives information 322 on a predetermined schedule. For example, number of surgical tools 304 may receive information 322 about every second. When information 322 is received, number of surgical tools 304 transmits information 322 to computer system 302 using link 305. In some illustrative embodiments, number of surgical tools 304 also identifies distance 332 between number of surgical tools 304 and tag 320. In such illustrative embodiments, number of surgical tools 304 also transmits distance 332 to computer system 302.

Surgical tool management process 308 receives information 322 and distance 332 from number of surgical tools 304. Surgical tool management process 308 then determines whether number of surgical tools 304 is to remain enabled for performing procedure 310. For example, surgical tool management process 308 may determine whether distance 332 is less than predetermined distance 344. In the event that information 322 is not received within a predetermined amount of time, distance 332 is more than predetermined distance 344, number of vital signs for patient 338 are no longer within limit 340, or another suitable condition occurs, surgical tool management process 308 ceases transmitting authorization 342. In some illustrative embodiments, surgical tool management process 308 may also transmit a deauthorization signal.

When number of surgical tools 304 is enabled, number of surgical tools 304 determines whether authorization 342 is received within period of time 346. When authorization is received within period of time 346, number of surgical tools 304 remains enabled. When authorization 342 is not received within period of time 346 and/or a deauthorization signal is received from computer system 302, number of surgical tools 304 is disabled. In one illustrative example, number of surgical tools 304 includes cutting tool 314. In such an illustrative embodiment, cutting tool 314 is disabled by cutting tool 314 causing cutting instrument 316 to retract into housing 318 such that cutting instrument 316 may not contact patient 306. Number of surgical tools 304 remains disabled until authorization 342 is received.

The illustration of computer system 302 in surgical tool management environment 300 is not meant to imply physical or architectural limitations to the manner in which different features may be implemented. Other components in addition to and/or in place of the ones illustrated may be used. Some components may be unnecessary in some illustrative embodiments. Also, the blocks are presented to illustrate some functional components. One or more of these blocks may be combined and/or divided into different blocks when implemented in different illustrative embodiments.

For example, in embodiments in which number of surgical tools 304 includes multiple surgical tools, surgical tool management process 308 may cause computer system 302 to transmit authorization 342 for all surgical tools in number of surgical tools 304 when one of number of surgical tools 304 is determined to be within predetermined distance 344 of patient 306 and/or that number of surgical tools 304 are to be used to perform procedure 310. In other illustrative embodiments, surgical tool management process 308 receives a user input from medical personnel when number of surgical tools 304 includes a surgical tool that is not to be used in performing procedure 310. Such a user input may include activation of a user interface component, input of a password, input of biometric credentials, or other suitable input.

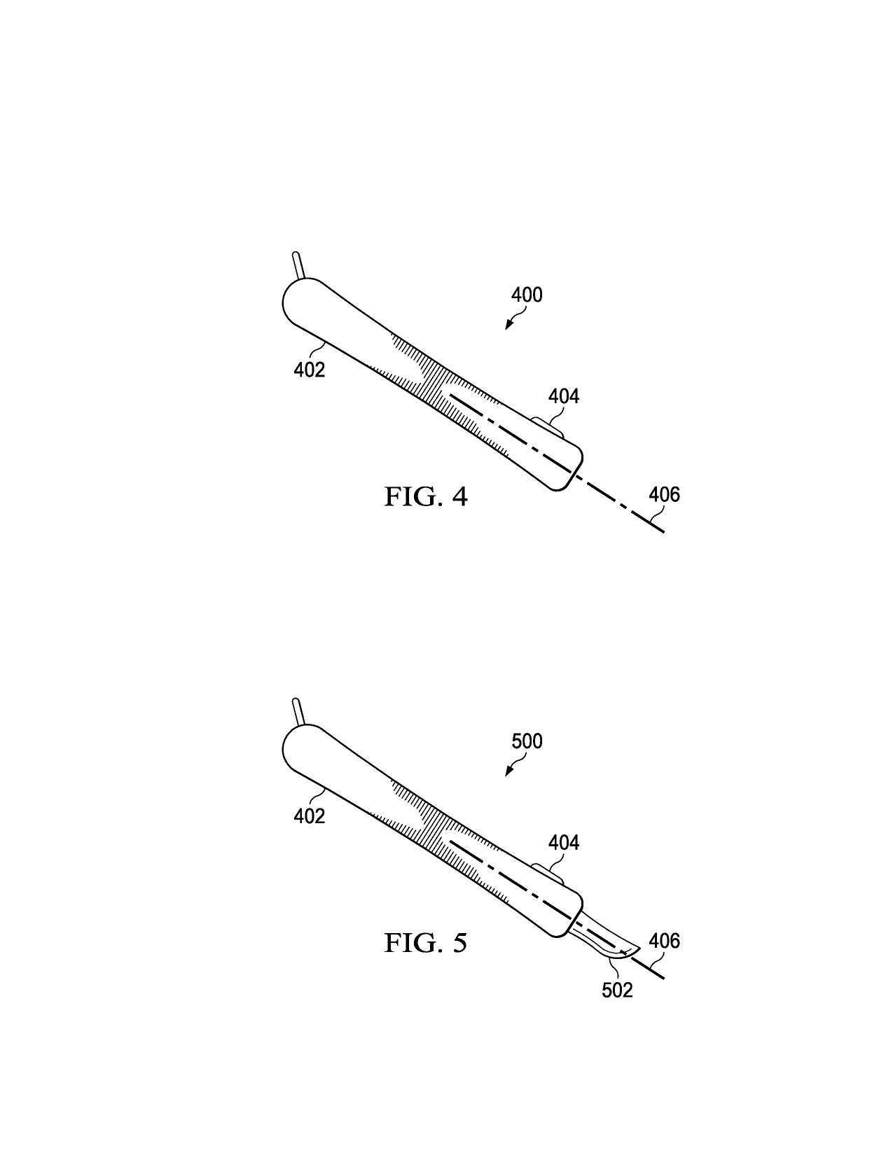

FIGS. 4 and 5 depict a surgical tool in accordance with an illustrative embodiment. FIG. 4 is an illustration of the surgical tool in a disabled state. FIG. 5 is an illustration of the surgical tool in an enabled state.

With reference now to FIG. 4, an illustration of a cutting tool in a disabled state is depicted in accordance with an illustrative embodiment. Cutting tool 400 is an example of cutting tool 314 in FIG. 3.

Cutting tool 400 is in a disabled state. In other words, cutting tool 400 may not be used to perform a procedure, such as procedure 310 in FIG. 3. In this illustrative embodiment, cutting tool 400 is disabled by the cutting instrument of cutting tool 400 being retracted within housing 402 of cutting tool 400. The cutting instrument travels along axis 406. Axis 406 is a longitudinal axis for housing 402 in this illustrative embodiment, along which the cutting instrument may travel into and out of housing 402. For example, the cutting instrument may travel through an opening in housing 402.

In this illustrative embodiment, receiver 404 is attached to housing 402. Receiver 404 is an example implementation of receiver 324 in FIG. 3. Receiver 404 may receive information from a tag, such as tag 320 in FIG. 3.

Looking now to FIG. 5, an illustration of a cutting tool in an enabled state is depicted in accordance with an illustrative embodiment. Cutting tool 500 is an example of cutting tool 400 after being enabled. In one illustrative embodiment, cutting tool 500 is enabled after receiving an authorization, such as authorization 342 in FIG. 3.

After receiving such an authorization over a link, such as link 305 in FIG. 3, cutting tool 500 is enabled. In this illustrative embodiment, cutting tool 500 is enabled by cutting instrument 502 being extended along axis 406. Cutting instrument 502 is extended such that cutting tool 502 may contact a patient, such as patient 306 in FIG. 3. Once cutting tool 500 is enabled, cutting instrument 502 remains extended until an authorization is not received within a period of time, such as period of time 346 in FIG. 3. When the authorization is not received within the period of time, cutting tool 500 is disabled. In this illustrative embodiment, cutting tool 500 is disabled by retracting cutting tool 502 along axis 406 until cutting tool 502 is located within housing 402.

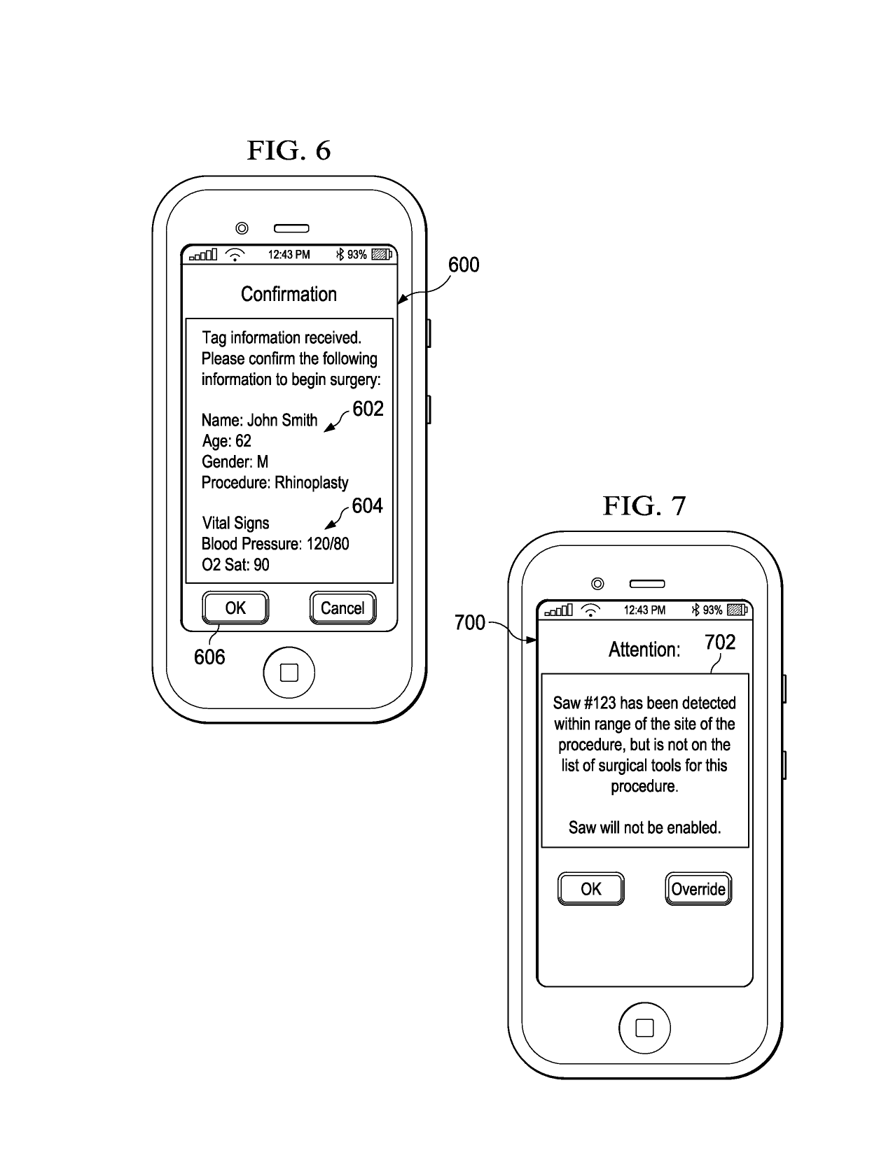

Turning now to FIG. 6, an illustration of a screenshot of a confirmation interface is depicted in accordance with an illustrative embodiment. Screenshot 600 may be generated by surgical tool management process 308 in FIG. 3.

Screenshot 600 includes information 602. Information 602 is an example implementation of information 322 in FIG. 3. In this illustrative example, information 602 includes a confirmation of patient identification information and the procedure to be performed. Screenshot 600 also includes vital signs 604. Vital signs 604 are example implementations of number of vital signs 338. Information 602 and vital signs 604 are displayed so medical personnel may verify the identity of the patient and the procedure to be performed. Further, medical personnel may verify that vital signs 604 are within desired limits. In the event that the medical personnel have confirmed information 602 and vital signs 604, medical personnel may activate button 606.

Looking now to FIG. 7, an illustration of a screenshot of a second confirmation interface is depicted in accordance with an illustrative embodiment. Screenshot 700 may be generated by surgical tool management process 308 in FIG. 3.

Screenshot 700 may be displayed when information is received from a surgical tool that is not to be used for performing the current procedure, according to a data source, such as data source 336 in FIG. 3. In another illustrative embodiment, screenshot 700 may be displayed when a distance from a tag is received from a surgical tool that is not to be used for performing the current procedure, and the surgical tool is within a predetermined distance of the tag, such as predetermined distance 344 in FIG. 3. Message 702 in screenshot 700 indicates that a saw has come within the particular distance of the tag and that the saw is not on the list of surgical tools to be used in performing the procedure. Thus, the saw is not to be enabled. However, in some illustrative embodiments, a medical professional may activate a user interface component to cause the saw to be enabled. For example, a medical professional may desire to use the saw when additional work on the patient is desired.

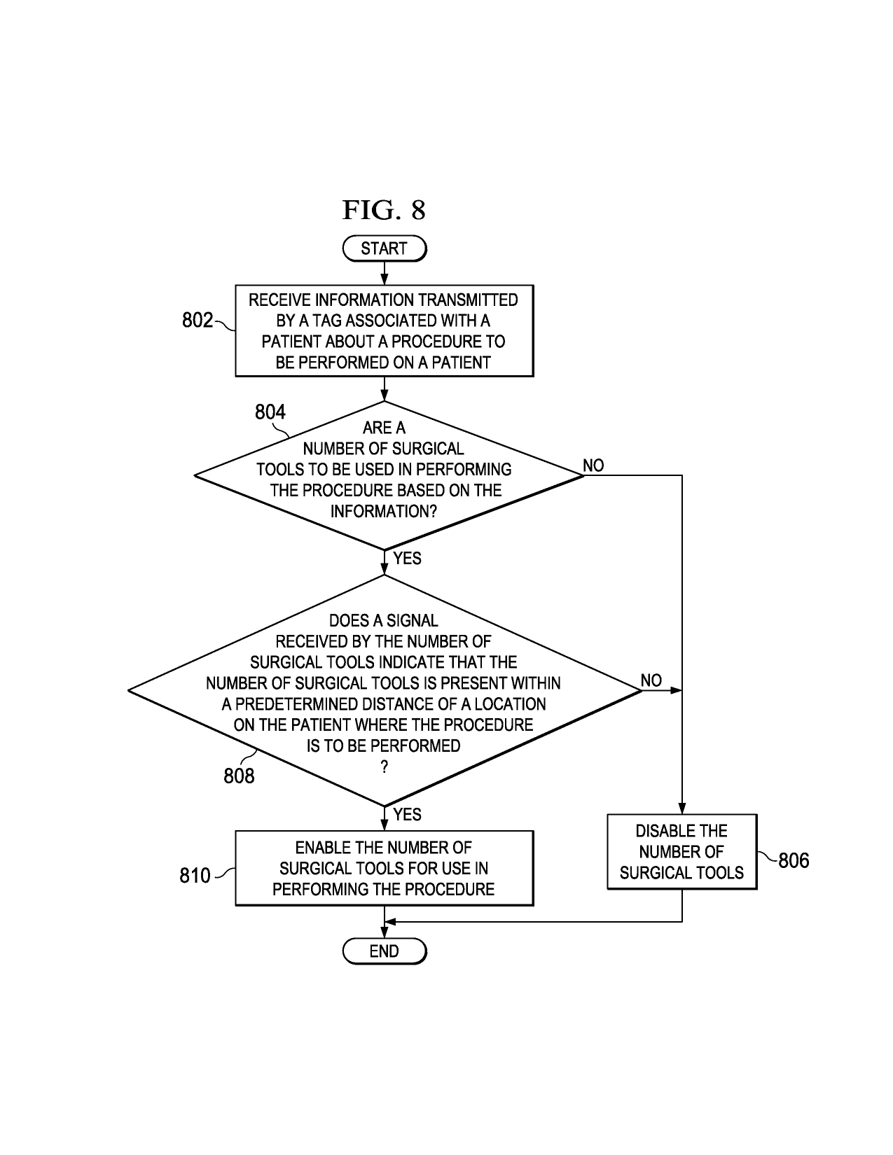

With reference now to FIG. 8, an illustration of a flowchart of a process for managing a number of surgical tools is depicted in accordance with an illustrative embodiment. The process may be performed by surgical tool management process 308 running on computer system 302 in FIG. 3.

The process begins by receiving information transmitted by a tag associated with a patient about a procedure to be performed on a patient (step 802). The procedure may be procedure 310 in FIG. 3. The information may include an identifier for the patient, a number of steps to be performed during the procedure, an identifier for the tag, and/or other suitable information.

The process then determines whether a number of surgical tools is to be used in performing the procedure based on the information (step 804). The number of surgical tools may be number of surgical tools 304 in FIG. 3. The information may include an identification of the surgical tools to be used in performing the procedure. In another illustrative embodiment, the information is an identifier for data about a procedure stored in a data source.

If the process determines that the number of surgical tools is not to be used in performing the procedure based on the information, the process disables the number of surgical tools (step 806) and terminates. In illustrative embodiments in which the number of surgical tools is already disabled, the process may cause the number of surgical tools to remain disabled. Disabling the surgical tools includes preventing the number of surgical tools from being used on the body of the patient. In one illustrative embodiment, the process disables a cutting tool by causing the cutting tool to retract the cutting instrument at least partially into a housing.

If the process determines that the number of surgical tools is to be used in performing the procedure based on the information, the process determines whether a signal received by the number of surgical tools indicates that the number of surgical tools is present within a predetermined distance of a location on the patient where the procedure is to be performed (step 808). The location on the patient is a point or an area at or near the site of the surgical procedure. The tag is removably attached to the patient at the point and/or in the area in these illustrative examples. If the process determines that the signal indicates that the number of surgical tools is not present within the predetermined distance of the location on the patient where the procedure is to be performed, the process proceeds to step 806.

If the process determines that the signal indicates that the number of surgical tools is present within the predetermined distance of the location on the patient where the procedure is to be performed at step 808, the process enables the number of surgical tools for use in performing the procedure (step 810). The process may enable the number of surgical tools by enabling a number of motors and/or other electrical circuits. In these illustrative embodiments, the process enables the number of surgical tools by causing the number of surgical tools to extend a cutting instrument from within a housing. The process terminates thereafter.

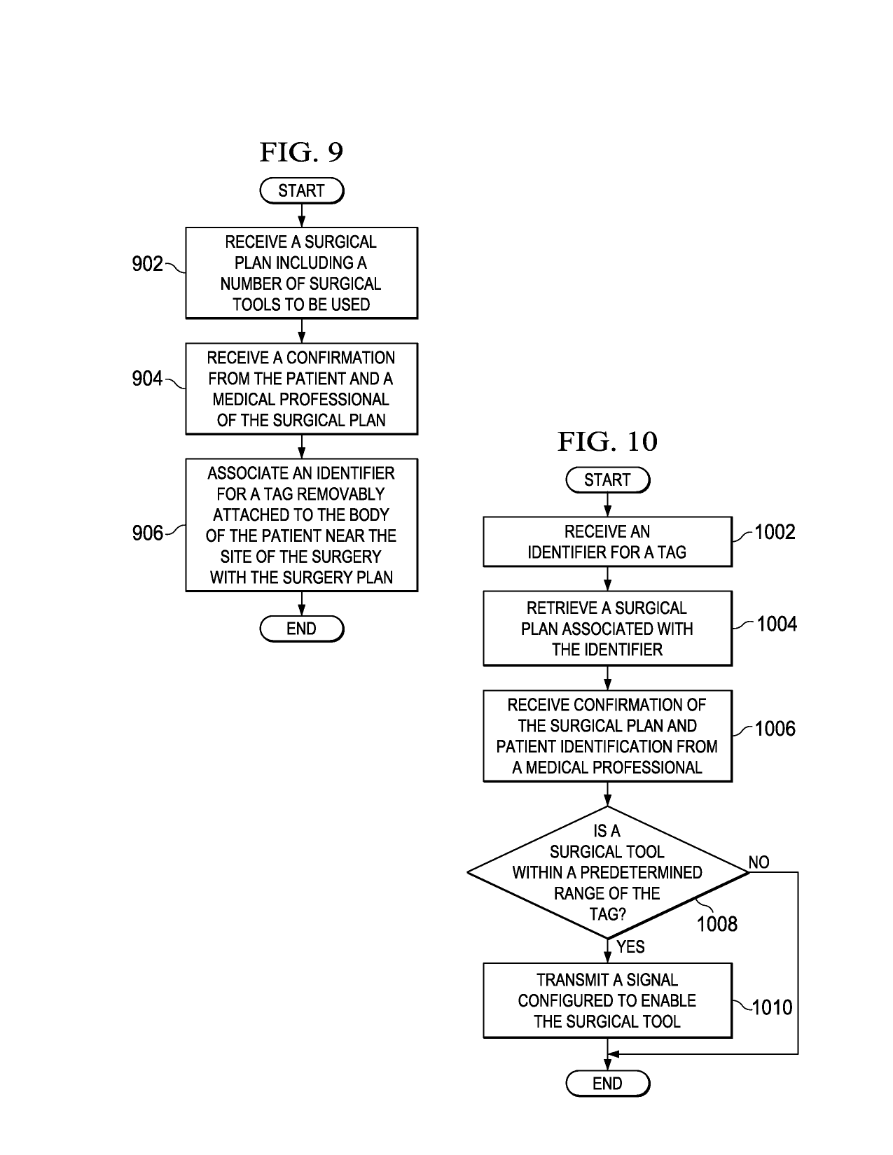

Turning now to FIG. 9, an illustration of a flowchart of a process for associating a tag having an identifier with a surgical plan is depicted in accordance with an illustrative embodiment. The process may be performed by surgery management process 308 running on computer system 302 in FIG. 3.

The process begins by receiving a surgical plan including a number of surgical tools to be used (step 902). The surgical plan may include an identification of the procedure to be performed, the steps to be taken in performing the procedure, the identification of the patient, and/or other suitable information. The number of surgical tools is an example of number of surgical tools 304 in FIG. 3.

The process then receives a confirmation from the patient and a medical professional of the surgical plan (step 904). The confirmation may be electronic. For example, the patient and/or the medical professional may electronically sign the surgical plan, provide a biometric authentication for the surgical plan, activate a button in a user interface confirming the surgical plan, or another suitable confirmation. The process then associates an identifier for a tag removably attached to the body of the patient near the site of the surgery with the surgery plan (step 906). The tag may be removably attached to the body of the patient by a medical professional. For example, a doctor may attach the tag to the patient adjacent to the location where an incision is to be made on the arm of a patient. The process terminates thereafter.

Looking now to FIG. 10, an illustration of a flowchart of a process for managing a surgical procedure is depicted in accordance with an illustrative embodiment. The process may be performed by surgical tool management process 308 running on computer system 302 in FIG. 3.

The process begins by receiving an identifier for a tag (step 1002). The process may receive the identifier for the tag using a receiver of a surgical tool, such as receiver 404 in FIG. 4. The identifier may indicate the identity of the patient and/or the surgical plan selected in step 902 in FIG. 9, and/or other suitable data. The process then retrieves the surgical plan associated with the identifier (step 1004).

The process then receives confirmation of the surgical plan and patient identification from a medical professional (step 1006). The confirmation may be received in the form of an activation of a user interface element, an audio statement spoken by the medical professional, an electronic signature, a biometric identification, or another suitable confirmation. The medical professional may then begin the surgical procedure.

The process then determines whether a surgical tool is within a predetermined range of the tag (step 1008). The predetermined range may be a range within which the receiver of the surgical tool may receive the identifier for the tag in one illustrative example. If the process determines that the surgical tool is not within the predetermined range of the tag, the process terminates. In other illustrative examples, the process returns to step 1008. The process may repeat step 1008 at a predetermined interval, continuously, or on another suitable schedule.

If the process determines that the surgical tool is within the predetermined range of the tag at step 1008, the process transmits a signal configured to enable the surgical tool (step 1010). The process terminates thereafter. In another illustrative example, the process returns to step 1008.

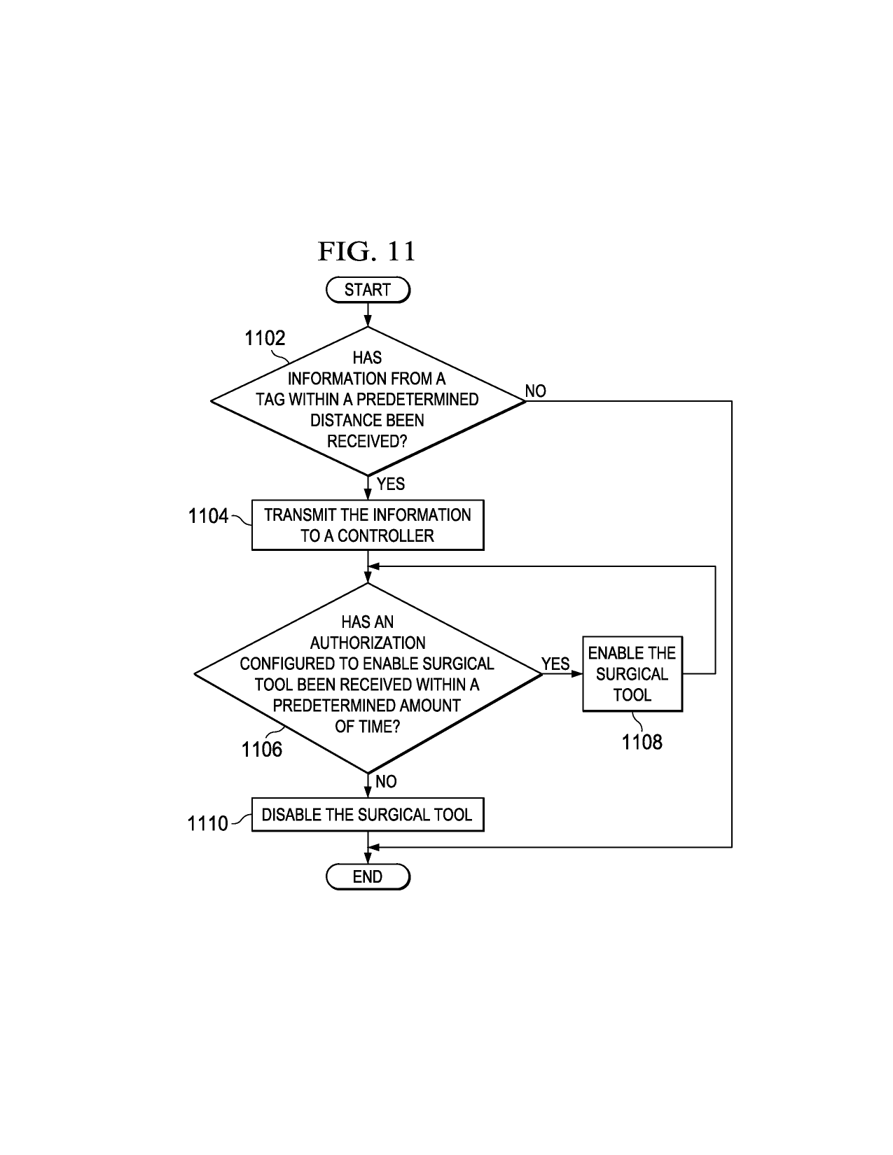

With reference now to FIG. 11, an illustration of a process for managing a surgical tool is depicted in accordance with an illustrative embodiment. The process may be performed by surgical tool management process 308 running on surgical tool 312 in FIG. 3.

The process determines whether information from a tag within a predetermined range has been received (step 1102). The information may be information 322 in FIG. 3. If the process determines that information from a tag within a predetermined range has not been received, the process terminates. In another illustrative embodiment, the process may repeat step 1102 on a predetermined interval, continuously, or another suitable schedule.

If the process determines that information has been received from a tag within the predetermined range at step 1102, the process transmits the information to a controller (step 1104). The controller may be computer system 302 in FIG. 3. The process then determines whether an authorization configured to enable the surgical tool has been received within a predetermined amount of time (step 1106). The authorization may be an example implementation of authorization 342 in FIG. 3. The authorization may be received using a wired or wireless transmission.

If the process determines that the authorization configured to enable the surgical tool has been received within the predetermined amount of time, the process enables the surgical tool (step 1108). In one illustrative example, a motor extends a cutting tool of a scalpel through an opening in a housing. In the event that the surgical tool is already enabled, the surgical tool may remain enabled. The process then returns to step 1106 to determine whether another authorization has been received within another predetermined period of time. If the process determines that the authorization configured to enable the surgical tool has not been received within the predetermined amount of time, the process disables the surgical tool (step 1110). In one illustrative example, the process disables the surgical tool by activating a motor that retracts the cutting tool of a scalpel through the opening in the housing. In the event that the surgical tool is already disabled, the surgical tool may remain disabled. The process terminates thereafter.

The different illustrative embodiments allow medical professionals to have confidence that the surgical procedure performed by the medical professional is performed on the intended patient in the intended location on the body of the patient. The surgical tools are enabled when the surgical tools are within a predetermined range of the tag at or near the site where the procedure is to be performed. In the event that a surgical tool is located at an undesired site and/or the surgical tool is not used in performing the procedure, the tool is disabled such that the tool may not cause undesired work to be performed on the body of the patient.

Thus, the different illustrative embodiments provide a method, computer program product, and apparatus for managing a number of surgical tools. Information is received about a procedure to be performed from a tag associated with a patient. A determination is then made as to whether a number of surgical tools is to be used in performing the procedure based on the information. Responsive to a determination that the number of surgical tools is to be used in performing the procedure, a determination is made as to whether the number of surgical tools is present within a predetermined distance of a location on the patient where the procedure is to be performed. Responsive to a determination that the number of surgical tools is within the predetermined distance of the location on the patient where the procedure is to be performed, the number of surgical tools is enabled for use in performing the procedure.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiment. The terminology used herein was chosen to best explain the principles of the embodiment, the practical application or technical improvement over technologies found in the marketplace, or to enable others of ordinary skill in the art to understand the embodiments disclosed here.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

What is claimed is:

1. A method for managing a number of surgical tools comprising:
receiving information transmitted by a tag associated with a patient about a procedure to be performed on the patient;
determining whether a number of surgical tools is to be used in performing the procedure based on the information;
responsive to a determination that the number of surgical tools is to be used in performing the procedure, determining whether a signal received by the number of surgical tools indicates that the number of surgical tools is present within a predetermined distance of a location on the patient where the procedure is to be performed; and
responsive to a determination that the signal received by the number of surgical tools indicates that the number of surgical tools is within the predetermined distance of the location on the patient where the procedure is to be performed, enabling the number of surgical tools for use in performing the procedure.

2. The method of claim 1, further comprising:
determining whether the number of surgical tools is no longer present within the predetermined distance of the location on the patient where the procedure is to be performed; and
responsive to a determination that the number of surgical tools is no longer present within the predetermined distance of the location on the patient where the procedure is to be performed, disabling the number of surgical tools.

3. The method of claim 1, wherein determining whether the signal received by the number of surgical tools indicates that the number of surgical tools is present within the predetermined distance of the location on the patient where the procedure is to be performed comprises:
determining whether the signal has been received from the tag within a period of time by a receiver associated with a surgical tool in the number of surgical tools;
determining whether the tag is within a predetermined distance of the number of surgical tools using the information; and
responsive to a determination that the signal has been received from the tag within the period of time by the receiver and that the tag is within the predetermined distance of the number of surgical tools using the signal, determining that the signal received by the number of tools indicates that the number of surgical tools are present within the predetermined distance of the location on the patient where the procedure is to be performed.

4. The method of claim 1, wherein the number of surgical tools comprises a cutting tool, and wherein enabling the number of surgical tools for use in performing the procedure comprises:
extending a cutting instrument for the cutting tool from a housing associated with the cutting tool.

5. The method of claim 2, wherein the number of surgical tools comprises a cutting tool, and wherein disabling the number of surgical tools for use in performing the procedure comprises:
retracting a cutting instrument for a bladed tool into a housing associated with the cutting tool.

6. The method of claim 1, wherein the information comprises the procedure to be performed and wherein determining whether the number of surgical tools is to be used in performing the procedure comprises:
determining whether the number of surgical tools is permitted to be used in performing the procedure in a data source.

7. The method of claim 1, wherein the information is selected from an identifier for the procedure to be performed, a number of steps to be performed for the procedure, and a patient identification.

8. The method of claim 3, wherein the signal is transmitted to a controller, and wherein enabling the number of surgical tools for use in performing the procedure comprises:
transmitting an authorization by the controller to the number of surgical tools, wherein the number of surgical tools is enabled upon receiving the authorization.

9. The method of claim 1, further comprising:
determining whether a number of vital signs for the patient are outside of a limit; and
responsive to a determination that the number of vital signs for the patient are outside of the limit, disabling the number of surgical tools.

10. A system comprising:
a number of surgical tools;
a controller configured to receive information transmitted by a tag associated with a patient about a procedure to be performed on the patient; determine whether a number of surgical tools is to be used in performing the procedure based on the information; determining whether a signal received by the number of surgical tools indicates that the number of surgical tools is present within a predetermined distance of a location on the patient where the procedure is to be performed responsive to a determination that the number of surgical tools is to be used in performing the procedure; and enable the number of surgical tools for use in performing the procedure responsive to a determination that the signal received by the number of surgical tools indicates that the number of surgical tools is within the predetermined distance of the location on the patient where the procedure is to be performed.

11. The system of claim 10, wherein the controller is further configured to determine whether the number of surgical tools is no longer present within the predetermined distance of the location on the patient where the procedure is to be performed; and responsive to a determination that the number of surgical tools is no longer present within the predetermined distance of the location on the patient where the procedure is to be performed, disable the number of surgical tools.

12. The system of claim 10, wherein the controller being configured to determine whether the signal received by the number of surgical tools is present within the predetermined distance of the location on the patient where the procedure is to be performed comprises the controller being configured to determine whether the signal has been received from the tag within a period of time by a receiver associated with a surgical tool in the number of surgical tools; determine whether the tag is within a predetermined distance of the number of surgical tools using the information; and responsive to a determination that the signal has been received from the tag within the period of time by the receiver and that the tag is within the predetermined distance of the number of surgical tools using the signal, determine that the signal received by the number of surgical tools indicates that the number of surgical tools are present within the predetermined distance of the location on the patient where the procedure is to be performed.

13. The system of claim 10, wherein the number of surgical tools comprises a cutting tool, and wherein the controller being configured to enable the number of surgical tools for use in performing the procedure comprises the number of surgical tools being configured to extend a cutting instrument for the cutting tool from a housing associated with the cutting tool.

14. The system of claim 11, wherein the number of surgical tools comprises a cutting tool, and wherein the controller being configured to disable the number of surgical tools for use in performing the procedure comprises the number of surgical tools being configured to retract a cutting instrument for a bladed tool into a housing associated with the cutting tool.

15. The system of claim 10, wherein the information comprises the procedure to be performed and wherein the controller being configured to determine whether the number of surgical tools is to be used in performing the procedure comprises the controller being configured to determine whether the number of surgical tools is permitted to be used in performing the procedure in a data source.

16. A computer program product comprising:
a computer readable storage medium;
program instructions, stored on the computer readable storage medium, for receiving information transmitted by a tag associated with a patient about a procedure to be performed on the patient;
program instructions, stored on the computer readable storage medium, for determining whether a number of surgical tools is to be used in performing the procedure based on the information;
program instructions, stored on the computer readable storage medium, for determining whether a signal received by the number of surgical tools indicates that the number of surgical tools is present within a predetermined distance of a location on the patient where the procedure is to be performed responsive to a determination that the number of surgical tools is to be used in performing the procedure; and
program instructions, stored on the computer readable storage medium, for enabling the number of surgical tools for use in performing the procedure responsive to a determination that the signal received by the number of surgical tools indicates that the number of surgical tools is within the predetermined distance of the location on the patient where the procedure is to be performed.

17. The computer program product of claim 16, further comprising:
program instructions, stored on the computer readable storage medium, for determining whether the number of surgical tools is no longer present within the predetermined distance of the location on the patient where the procedure is to be performed; and
program instructions, stored on the computer readable storage medium, for disabling the number of surgical tools responsive to a determination that the number of surgical tools is no longer present within the predetermined distance of the location on the patient where the procedure is to be performed.

18. The computer program product of claim 16, wherein the program instructions, stored on the computer readable storage medium, for determining whether the signal received by the number of surgical tools indicates that the number of surgical tools is present within the predetermined distance of the location on the patient where the procedure is to be performed comprise:
program instructions, stored on the computer readable storage medium, for determining whether the signal has been received from the tag within a period of time by a receiver associated with a surgical tool in the number of surgical tools;
program instructions, stored on the computer readable storage medium, for determining whether the tag is within a predetermined distance of the number of surgical tools using the information; and
program instructions, stored on the computer readable storage medium, for determining that the signal received by the number of surgical tools indicates that the number of surgical tools are present within the predetermined distance of the location on the patient where the procedure is to be performed responsive to a determination that the signal has been received from the tag within the period of time by the receiver and that the tag is within the predetermined distance of the number of surgical tools using the signal.

19. The computer program product of claim 16, wherein the number of surgical tools comprise a cutting tool, and wherein the program instructions, stored on the computer readable storage medium, for enabling the number of surgical tools for use in performing the procedure comprise:
program instructions, stored on the computer readable storage medium, for extending a cutting instrument for the cutting tool from a housing associated with the cutting tool.

20. The computer program product of claim 17, wherein the number of surgical tools comprise a cutting tool, and wherein the program instructions, stored on the computer readable storage medium, for disabling the number of surgical tools for use in performing the procedure comprise:
program instructions, stored on the computer readable storage medium, for retracting a cutting instrument for a bladed tool into a housing associated with the cutting tool.
